(19) United States
(12) Patent Application Publication
(10) Pub. No.: US 2021/0378702 A1
(43) Pub. Date: Dec. 9, 2021

(54) PUNCTURE NEEDLE GRIPPING DEVICE AND PUNCTURE ROBOT HAVING PUNCTURE NEEDLE GRIPPING DEVICE

(71) Applicant: NATIONAL UNIVERSITY CORPORATION OKAYAMA UNIVERSITY, Okayama-shi (JP)

(72) Inventors: Tetsushi KAMEGAWA, Okayama-shi (JP); Takayuki MATSUNO, Okayama-shi (JP); Takao HIRAKI, Okayama-shi (JP); Yuta MIKI, Okayama-shi (JP); Keiji TANIMOTO, Okayama-shi (JP)

(21) Appl. No.: 17/331,024

(22) Filed: May 26, 2021

(30) Foreign Application Priority Data
Jun. 5, 2020 (JP) 2020-98854

(51) Int. Cl. A61B 17/34; A61B 34/30

(52) U.S. Cl. CPC A61B 17/34; A61B 2017/0042; A61B 34/30

(57) ABSTRACT

In a puncture needle gripping device that grips a puncture needle that punctures a target in a patient's body and a puncture robot having the puncture needle gripping device, a puncture needle gripping device that can release the puncture needle gripped by the puncture needle gripping device by a remote control, and a puncture robot having the puncture needle gripping device are provided. A puncture needle gripping device includes a first finger and a second finger that pinches a proximal end of a puncture needle therebetween, and a releasing mechanism that grips the puncture needle by pinching the proximal end of the puncture needle between the first finger and the second finger and releases the puncture needle by separating the first finger and the second finger from each other by remote control.

### CROSS REFERENCE TO RELATED APPLCATIONS

The present invention claims priority under 35 U.S.C. § 119 to Japanese Patent Application No. 2020-98854, filed on Jun. 5, 2020, which the entire content of which is incorporated herein by reference.

### BACKGROUND OF THE INVENTION

### Field of the Invention

The present invention relates to a puncture needle gripping device for gripping a puncture needle, and particularly, to a puncture needle gripping device having a releasing mechanism that enables a puncture needle to be immediately released in an emergency and a puncture robot having the puncture needle gripping device.

Description of Related Art

The present inventors have developed a puncture robot used to puncture a lesion such as a tumor with a puncture needle under X-ray fluoroscopy by Computed Tomography (CT) (for example, refer to International Publication No. WO 2017/065016 and International Publication No. WO2018/207498). In this puncture robot, a puncture needle is mounted on a distal end of an arm having a predetermined length, and the puncture needle moves forward in an axial direction via the arm so that an affected portion which is a target in a patient's body can be punctured by the puncture needle.

When the puncture needle is mounted on the distal end of the arm, the puncture needle is mounted on a needle support portion provided at the distal end of the arm. In the needle support portion, the puncture needle can be detachably mounted by pinching a proximal end portion of the puncture needle between a first needle gripping arm and a second needle gripping arm provided so as to extend forward from a base of the needle support portion (for example, refer to International Publication No. WO 2018/207498 (FIG. 4)).

In particular, the first needle gripping arm and the second needle gripping arm are rotatably attached to the base of the needle support portion on a proximal end side, respectively, interposes the proximal end portion of the puncture needle between the respective distal end sides, are locked by a snap lock installed on the distal ends of the first needle gripping arm and the second needle gripping arm, and thus, can securely mount the puncture needle on the distal ends of the arm.

### PRIOR ART LITERATURE

### Patent Document

[Patent Document 1] International Publication No. 2017/065016

[Patent Document 2] International Publication No. 2018/207498

### SUMMARY OF THE INVENTION

In the above-mentioned puncture robot, the puncture needle is firmly gripped to prevent the puncture needle from falling in any case. However, depending on a use situation of the puncture robot, there is a risk that the puncture needle would have to be removed from the arm immediately.

In particular, since the puncture robot of the present invention has been developed for a purpose of allowing a practitioner to operate as far away from CT as possible, the practitioner also works away from the body of the puncture robot during a treatment. Accordingly, there is a problem that an operation of removing the puncture needle from the arm could not be performed instantly.

Therefore, in the present invention, in a puncture needle gripping device that grips a puncture needle that punctures a target in a patient's body, the puncture needle gripping device is provided with a releasing mechanism that releases the gripped puncture needle by a remote control.

Further, the puncture needle gripping device of the present invention also has the following characteristics.

(1) A first finger and a second finger are provided to pinch a proximal end of the puncture needle therebetween, the puncture needle is gripped by pinching the proximal end of the puncture needle between the first finger and the second finger, and the releasing mechanism releases the puncture needle by separating the first finger and the second finger from each other.

(2) The releasing mechanism is provided with an actuator that maintains a pinching state in which the proximal end of the puncture needle is pinched between the first finger and the second finger.

(3) The releasing mechanism is provided with a detector which detects that a state is in the pinching state, and the actuator is operated by the detector detecting that the state is in the pinching state.

(4) The first finger is mounted on a first substrate that slides along a linear guide, and the detector detects that the first substrate reaches a position where the state is in the pinching state.

(5) The releasing mechanism is provided with a biasing means, and the first substrate is moved to the position where the state is in the pinching state against a biasing force of the biasing means.

(6) The biasing means is a spring, the spring is mounted on a spring rod provided in parallel with the linear guide, a spring stopper biased by the spring is slidably mounted on the spring rod, and the spring stopper is attached to the first substrate.

(7) A rod of the actuator is connected to the spring stopper.

(8) A first rack is provided in the first substrate, a second substrate slidable along the linear guide is provided with a second rack that is interlocked and connected to the first rack via a pinion, and the second finger is mounted on the second substrate.

Further, a puncture robot of the present invention includes the puncture needle gripping device provided at a distal end of a longitudinal arm, and a target in a patient' body is punctured by the puncture needle by moving the arm forward in an axial direction of the puncture needle gripped by the puncture needle gripping device. Further, the arm is provided with a spare switch for performing the remote control, and the remote control is an emergency stop operation.

According to the present invention, the puncture needle gripping device for gripping the puncture needle is provided with the releasing mechanism for releasing the puncture needle by remote control, and thus, a releasing operation of the puncture needle can be performed instantly without causing a time loss.

### BRIEF DESCRIPTION OF THE DRAWINGS

FIG. 1 is an explanatory diagram of a puncture robot;

FIG. 2 is an external explanatory view of a puncture needle gripping device;

FIG. 3 is an external explanatory view of the puncture needle gripping device;

FIG. 4 is an assembled exploded view of the puncture needle gripping device;

FIG. 5 is an explanatory diagram of a releasing mechanism in a puncture needle gripping device; and

FIG. 6A and FIG. 6B are operation explanatory views of the puncture needle gripping device.

### DETAILED DESCRIPTION OF PREFERRED EMBODIMENTS

A puncture needle gripping device of the present invention is mounted on a distal end of an arm of a puncture robot provided with a longitudinal arm, and the puncture needle gripping device grips a puncture needle. In the puncture robot, the puncture needle is detachably mounted on the distal end of the arm via the puncture needle gripping device, and thus, a target in the patient's body can be punctured by the puncture needle by moving the puncture needle forward together with the arm in an axial direction.

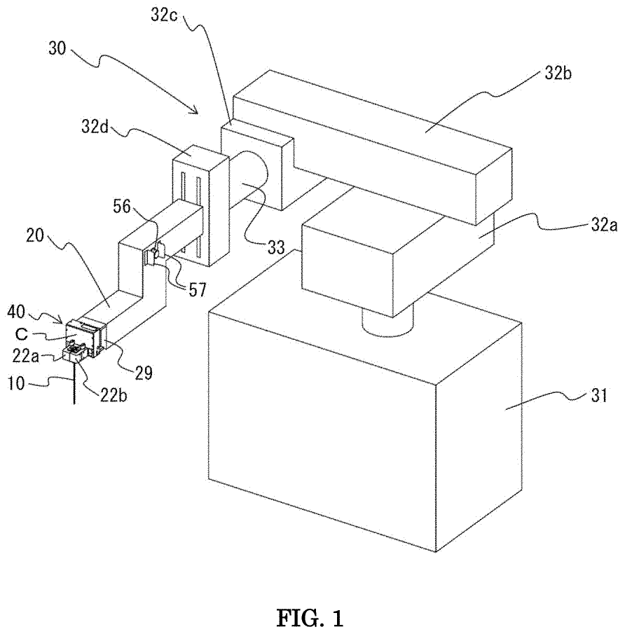

Specifically, as illustrated in FIG. 1, a puncture robot includes an arm 20 and a main body 30, in which a puncture needle gripping device 40 that detachably pinches a proximal end of a puncture needle 10 is mounted on a distal end of the arm 20, and the main body 30 has an adjustment mechanism that adjusts a position and an inclination of the puncture needle 10 via the arm 20.

The main body 30 has a rectangular base 31, and a first translation mechanism 32a that controls a parallel movement in a first horizontal direction is provided on an upper surface of the base 31. A second translation mechanism 32b that controls a parallel movement in a second horizontal direction orthogonal to the first horizontal direction is provided on an upper surface of the first translation mechanism 32a. A first rotation mechanism 32c having a rotation rod 33 extended in parallel with the first horizontal direction is provided at a distal end portion of the second translation mechanism 32b, and can control a rotation of the rotation rod 33 around the central axis of the rotation rod 33. A forward/rearward movement mechanism 32d for moving the arm 20 forward or rearward in an extension direction of the puncture needle 10 is provided at a distal end of the rotation rod 33. In particular, the forward/rearward movement mechanism 32d is attached to the rotation rod 33 via a rotating shaft (not illustrated) orthogonal to an extension direction of the rotation rod 33, and a second rotation mechanism (not illustrated) for rotating the forward/rearward movement mechanism 32d is provided around the rotating shaft. An inclination of the puncture needle 10 can be freely controlled by the second rotation mechanism and the first rotation mechanism 32c. The arm 20 constitutes a parallel link by an upper arm and a lower arm (not illustrated), and by operating the arm 20 forward by the forward/rearward movement mechanism 32d to operate the puncture needle 10 forward in the axial direction in a state where a forward/rearward direction of the arm 20 by the forward/rearward movement mechanism 32d is parallel with an axial direction of the puncture needle 10, the target in the patient's body can be punctured by the puncture needle 10. Further, although not illustrated, a raising/lowering mechanism for raising or lowering the first translation mechanism 32a in a vertical direction is provided inside the base 31, and by controlling 6 degrees of freedom of the raising/lowering mechanism, the position and inclination of the puncture needle 10 can be freely controlled.

A main portion of the present invention is the puncture needle gripping device 40 provided at the distal end of the arm 20, and thus, hereinafter, the puncture needle gripping device 40, a method of mounting the puncture needle 10 on the puncture needle gripping device 40, and a method of releasing the puncture needle 10 will be described in detail.

As illustrated in FIG. 1, the puncture needle gripping device 40 is mounted on the distal end of the arm 20, and the puncture needle 10 is detachably mounted on the puncture needle gripping device 40.

An instantaneous puncture mechanism 29, which instantly moves the puncture needle gripping device 40 forward in the axial direction of the puncture needle 10 by about 10 mm by an air cylinder (not illustrated), is provided at the distal end of the arm 20, the puncture needle gripping device 40 is mounted on the instantaneous puncture mechanism 29, and thus, the puncture needle 10 can be moved instantly together with the puncture needle gripping device 40.

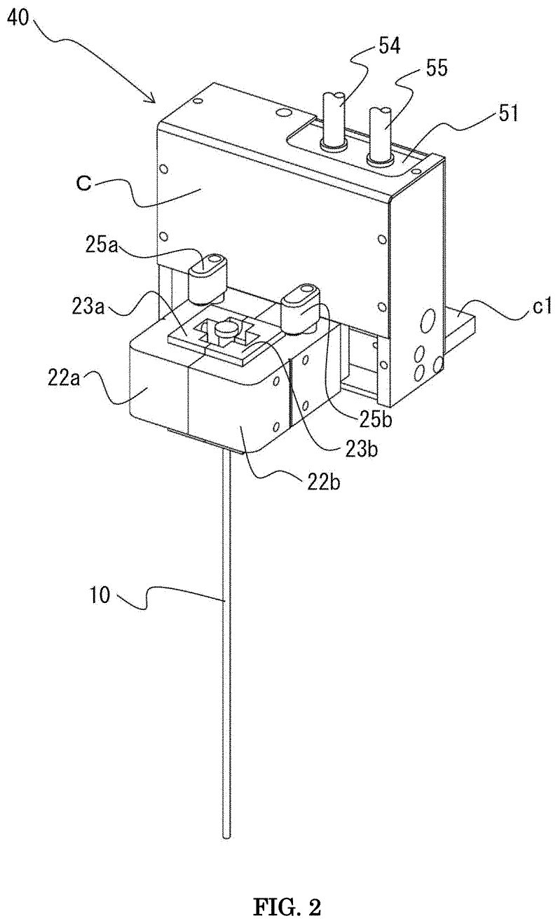

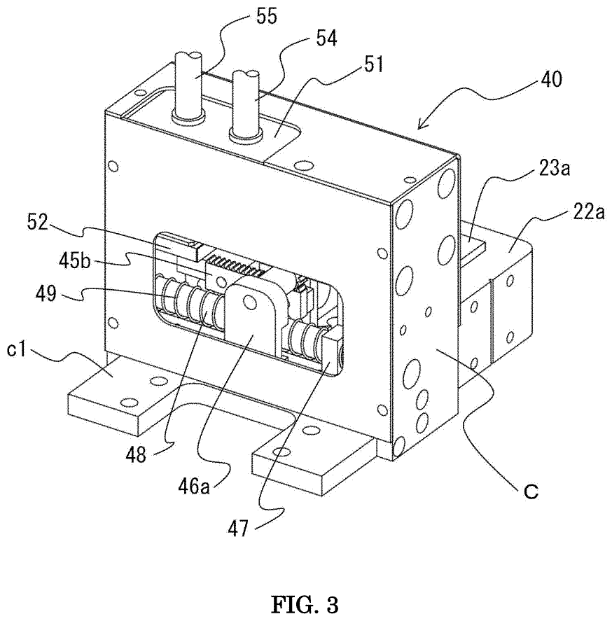

As illustrated in FIG. 2 and FIG. 3, the puncture needle gripping device 40 includes a mechanical portion covered with a rectangular casing C and a first finger 22a and a second finger 22b that protrude forward from the mechanical portion and pinches the proximal end of the puncture needle 10 therebetween. A releasing mechanism is provided in the mechanical portion covered with the casing C as described below. In FIG. 2 and FIG. 3, as described below, a reference numeral 54 indicates a supply pipe of compressed air for driving an actuator 51 provided in the casing C and a reference numeral 55 is an exhaust pipe. Further, in FIG. 2 and FIG. 3, a reference numeral cl is a bottom plate of the casing C, which is provided so as to protrude rearward of the puncture needle gripping device 40 and serves as an attachment portion to the instantaneous puncture mechanism 29.

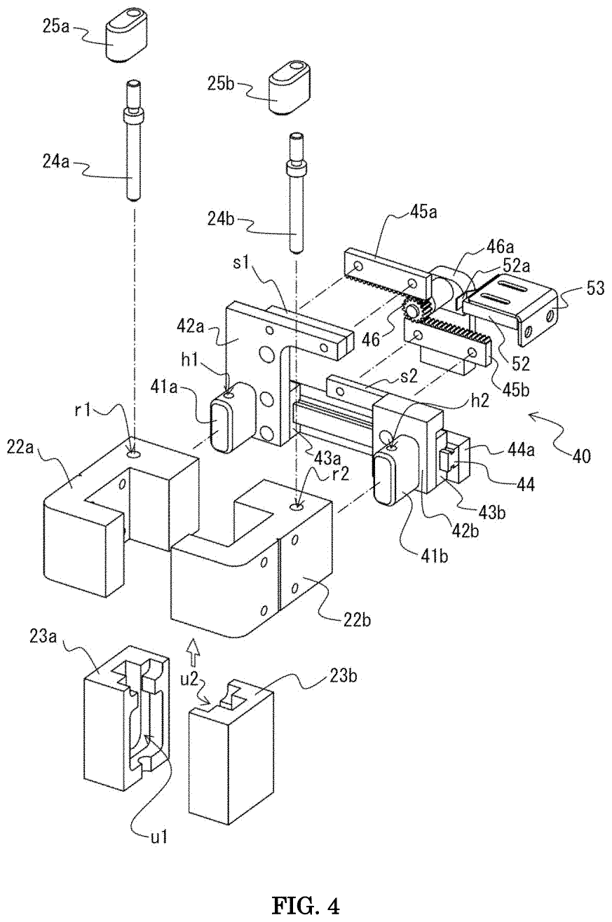

As illustrated in FIG. 2 and FIG. 4, a first attachment 23a having a first fitting recessed portion u1 fitted with a side surface of the proximal end of the puncture needle 10 can be detachably mounted on the first finger 22a, and a second attachment 23b having a second fitting recessed portion u2 fitted with the side surface of the proximal end of the puncture needle 10 can be detachably mounted on the second finger 22b.

In the first finger 22a on which the first attachment 23a is mounted and the second finger 22b on which the second attachment 23b is mounted, the first fitting recessed portion u1 of the first attachment 23a and the second fitting recessed portion u2 of the second attachment 23b are disposed to face each other and abut against each other, and thus, the first fitting recessed portion u1 and the second fitting recessed portion u2 can pinch the proximal end of the puncture needle 10 therebetween while fitting with the proximal end of the puncture needle 10.

By providing the first attachment 23a and the second attachment 23b, it is possible to mount various puncture needles having different proximal end shapes simply by making the first attachment 23a and the second attachment 23b have shapes corresponding those of the puncture needles, and versatility can increase.

As illustrated in FIG. 4, the first finger 22a is provided with a fitting hole (not illustrated) for fitting with a first protruding piece 41a provided to protrude forward, and the second finger 22b is provided with a fitting hole (not illustrated) for fitting with a second protruding piece 41b provided to protrude forward.

The first protruding piece 41a and the second protruding piece 41b are provided with a first through hole h1 and a second through hole h2 penetrating in an up-down direction, respectively. The first finger 22a is provided with a first communication hole r1 that communicates with the first through hole h1 in the up-down direction when being fitted to the first protruding piece 41a, and the second finger 22b is provided with a second communication hole r2 that communicates with the second through hole h2 in the up-down direction when being fitted to the second protruding piece 41b.

The first finger 22a is fitted to and mounted on the first protruding piece 41a to communicate the first through hole h1 and the first communication hole r1 with each other, and thus, the first finger 22a can be detachably mounted by inserting a first locking pin 24a into the first communication hole r1 from above the first communication hole r1. Further, the second finger 22b is fitted to and mounted on the second protruding piece 41b to communicate the second through hole h2 and the second communication hole r2 with each other, and thus, the second finger 22b can be detachably mounted by inserting a second locking pin 24b into the second communication hole r2 from above the second communication hole r2. A first knob piece 25a and a second knob piece 25b are provided at end portions of the first locking pin 24a and the second locking pin 24b, respectively.

In this way, the first finger 22a and the second finger 22b are easily detachable via the first locking pin 24a and the second locking pin 24b, respectively. Accordingly, in the puncture robot that needs to be sterilized and used, the first finger 22a on which the first attachment 23a is mounted, the second finger 22b on which the second attachment 23b is mounted, the first locking pin 24a, and the second locking pin 24b can be separated from the puncture needle gripping device 40 and sterilized individually or made disposable. In addition, the mechanical portion, the arm 20, and the main body 30 of the puncture needle gripping device 40 from which the first finger 22a and the second finger 22b are removed are sterilized by covering with a sterilized sheet such as a drape (not illustrated).

As illustrated in FIG. 4, the first protruding piece 41a on which the first finger 22a is mounted is attached to a surface of a flat plate-shaped first substrate 42a, and the second protruding piece 41b on which the second finger 22b is mounted is attached to a surface of a flat plate-shaped second substrate 42b.

As illustrated in FIG. 4, a first sliding body 43a, which is slidably mounted on a linear guide 44 extending in a horizontal direction, is attached to a back surface of the first substrate 42a, and the first substrate 42a is slidable along the linear guide 44. Further, a second sliding body 43b, which is slidably mounted on the linear guide 44, is attached to a back surface of the second substrate 42b, and the second substrate 42b is slidable along the linear guide 44. That is, the first protruding piece 41a and the second protruding piece 41b can oscillate in the horizontal direction along the linear guide 44, and the first finger 22a and the second finger 22b can oscillate in the horizontal direction.

As illustrated in FIG. 4, a first rack 45a is attached to an upper edge of the first substrate 42a, an attachment piece s2 is provided to protrude along an upper edge of the second substrate 42b, and the second rack 45b is mounted on the attachment piece s2 so as to face the first rack 45a. In FIG. 4, a reference numeral s1 indicates a spacer interposed between the first substrate 42a and the first rack 45a.

The first rack 45a and the second rack 45b are interlocked and connected to each other via a pinion 46, and when any one of the first substrate 42a or the second substrate 42b moves, the other thereof can move by an equidistant distance in the opposite direction. Accordingly, the first finger 22a mounted on the first protruding piece 41a and the second finger 22b mounted on the second protruding piece 41b can be opened and closed.

In the following, for convenience of explanation, a movement of the first substrate 42a and the second substrate 42b in a direction in which the first finger 22a and the second finger 22b are brought close to each other is referred to as a “close-direction movement”, and conversely, a movement of the first substrate 42a and the second substrate 42b in a direction in which the first finger 22a and the second finger 22b are separated from each other is referred to as an “open-direction movement”.

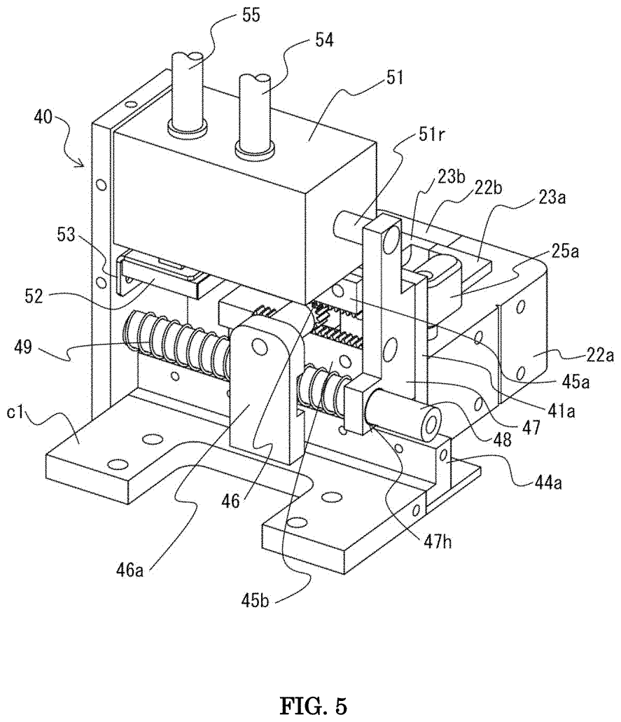

As illustrated in FIG. 5, the pinion 46 is rotatably mounted on a pinion support plate 46a erected on a bottom plate c1 of the casing C. In FIG. 4 and FIG. 5, a reference numeral 44a indicates a linear guide support in which a linear guide 44 is mounted on a front surface.

FIG. 5 is an explanatory view of the releasing mechanism of the puncture needle gripping device 40 in which a portion of the casing C is omitted, and as illustrated in FIG. 5, a flat plate-shaped spring stopper 47 is attached to a back surface of the first substrate 42a.

The spring stopper 47 is provided with a through hole 47h through which passes the spring rod 48 provided in parallel with the linear guide 44, and the spring stopper 47 is slidable with respect to the spring rod 48.

A spring 49 is attached to the spring rod 48 so as to pass through the spring rod 48, and the spring 49 biases the spring stopper 47 to move the first substrate 42a in the opening direction. That is, the spring 49 is a biasing means. Further, when the first substrate 42a is moved in the opening direction by the spring 49, the second substrate 42b interlocked and connected via the pinion 46 is also moved in the opening direction.

Further, a distal end of a rod 51r of the actuator 51 provided in the casing C is connected to the spring stopper 47.

The actuator 51 is operated using compressed air as a power source, a state becomes a locked state in which a forward or rearward movement of the rod 51r is stopped when the actuator 51 is in an operating state, while the state becomes an unlocked state and the rod 51r can be moved forward or rearward freely when the operating state is released.

In particular, in the unlocked state, the rod 51r is moved forward by biasing of the spring 49 against the spring stopper 47 to which the distal end of the rod 51r is connected.

As illustrated in FIG. 4 and FIG. 5, a detection of the actuator 51 in the operating state is performed by a limit switch 52 provided in the casing C.

In the limit switch 52, the first substrate 42a and the second substrate 42b move in the closed direction, and thus, when a state becomes a state where the first finger 22a and the second finger 22b abut against each other, that is, a state where the proximal end of the puncture needle 10 is pinches between the first finger 22a and the second finger 22b, the distal end of the first rack 45a attached to the first substrate 42a comes into contact with a detection lever 52a of the limit switch 52 and is mounted at a position at which the limit switch 52 is turned on. In particular, for this position adjustment, the limit switch 52 is mounted in the casing C via an attachment guide 53. That is, the limit switch 52 is a detector that detects that the proximal end of the puncture needle 10 is pinched between the first finger 22a and the second finger 22b.

An on signal of the limit switch 52 is input to the actuator 51, and thus, the actuator 51 is operated and the state becomes the locked state.

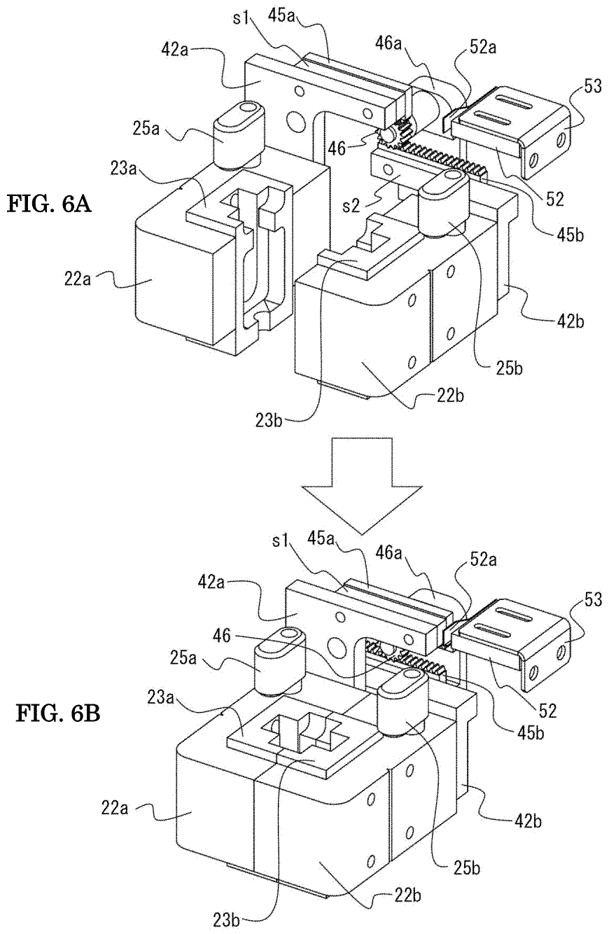

That is, in the puncture needle gripping device 40, as illustrated in FIG. 6A, the first substrate 42a and the second substrate 42b are moved in the closed direction against the biasing of the spring 49 from a state where the first finger 22a mounted on the first protruding piece 41a and the second finger 22b mounted on the second protruding piece 41b are separated from each other by the biasing of the spring 49, and as illustrated in FIG. 6B, when the first finger 22a and the second finger 22b abut against each other, the distal end of the first rack 45a provided on the first substrate 42a comes into contact with the detection lever 52a of the limit switch 52 to operate the actuator 51, a pinching state of the puncture needle 10 between the first finger 22a and the second finger 22b is maintained, and the puncture needle 10 is gripped.

In this way, the proximal end of the puncture needle 10 is pinched and fixed by the first finger 22a and the second finger 22b and the pinching state is maintained only by moving the first finger 22a and the second finger 22b. Therefore, there is no possibility that the puncture needle 10 is inadequately pinched due to an operation error, and the puncture needle 10 can be reliably gripped.

In the puncture needle gripping device 40, the actuator 51 is unlocked simply by inputting an off signal to the actuator 51, and the first substrate 42a and the second substrate 42b are moved in the opening direction by the biasing of the spring 49, the first finger 22a and the second finger 22b are separated from each other, and thus, the puncture needle 10 can be released. In this way, the actuator 51 and the spring 49 can be used to release the puncture needle by separating the first finger 22a and the second finger 22b from each other, and the releasing mechanism is constituted by the limit switch 52 that generates an activation signal of the actuator 51.

Since the input of the off signal to the actuator 51 is merely a signal input, a remote control is possible. For example, by providing a release switch in an operation panel (not illustrated) operating the puncture robot, the puncture needle 10 can be instantly released from the puncture needle gripping device 40 by remote control. Alternatively, based on a signal input from a body motion sensor (not illustrated) that not only inputs the off signal to the actuator 51 by the release switch but also detects a movement of the patient's body to be punctured by the puncture needle 10, a sensor other than these, or an external sensor, the off signal to the actuator 51 may be input. In particular, when linked to the body motion sensor, it is possible to respond faster than a practitioner performing the puncture with the puncture needle 10, and certainty can be further improved.

Further, the off signal may be input to the actuator 51 by operating an emergency stop switch instead of the release switch, or the puncture needle may be instantly released from the puncture needle gripping device 40 as a remote control by the emergency stop operation.

In addition to providing the release switch or emergency stop switch in the operation panel, as illustrated in FIG. 1, a spare switch 56 for emergency stop may be provided in the arm 20, and by operating the spare switch 56, the puncture needle 10 may be instantly released from the puncture needle gripping device 40 as a remote control.

As illustrated in FIG. 1, when the arm 20 is covered with a sterilized sheet such as a drape, a protective wall 57 is provided around the spare switch 56 so as to prevent unintended operation of the spare switch 56 due to an interference between the seat and the spare switch 56. In the present embodiment, the protective wall 57 is provided as a pair of two protective walls with the spare switch 56 interposed therebetween.

### DESCRIPTION OF SIGN

10 puncture needle

20 arm

22a first finger

22b second finger

23a first attachment

23b second attachment

24a first locking pin

24b second locking pin

25a first knob piece

25b second knob piece

u1 first fitting recessed portion

u2 second fitting recessed portion

r1 first communication hole

r2 second communication hole

29 instantaneous puncture mechanism

30 main body

40 puncture needle detachment mechanism

41a first protruding piece

41b second protruding piece

42a first substrate

42b second substrate

43a first sliding body

43b second sliding body

44 linear guide

44a linear guide support

45a first rack

45b second rack

46 pinion

46a pinion support plate

47 spring stopper

47h through hole

48 spring rod

49 spring

51 actuator

51r rod

52 limit switch

52a detection lever

53 attachment guide

s1 spacer

s2 attachment piece

h1 first through hole

h2 second through hole

C casing

What is claimed is:

1. A puncture needle gripping device that grips a puncture needle that punctures a target in a patient's body, which comprises:
a releasing mechanism that releases the puncture needle gripped by the puncture needle gripping device by a remote control.

2. The puncture needle gripping device according to claim 1, wherein a first finger and a second finger are provided to pinch a proximal end of the puncture needle therebetween, the puncture needle is gripped by pinching the proximal end of the puncture needle between the first finger and the second finger, and
the releasing mechanism releases the puncture needle by separating the first finger and the second finger from each other.

3. The puncture needle gripping device according to claim 2, wherein the releasing mechanism is provided with an actuator that maintains a pinching state in which the proximal end of the puncture needle is pinched between the first finger and the second finger.

4. The puncture needle gripping device according to claim 3, wherein the releasing mechanism is provided with a detector which detects that a state is in the pinching state, and
the actuator is operated by the detector which detects that the state is in the pinching state.

5. The puncture needle gripping device according to claim 4, wherein the first finger is mounted on a first substrate that slides along a linear guide, and
the detector detects that the first substrate reaches a position where the state is in the pinching state.

6. The puncture needle gripping device according to claim 5, wherein the releasing mechanism is provided with a biasing means, and the first substrate is moved to the position where the state is in the pinching state against a biasing force of the biasing means.

7. The puncture needle gripping device according to claim 6, wherein the biasing means is a spring, the spring is mounted on a spring rod provided in parallel with the linear guide, a spring stopper biased by the spring is slidably mounted on the spring rod, and the spring stopper is attached to the first substrate.

8. The puncture needle gripping device according to claim 7, wherein a rod of the actuator is connected to the spring stopper.

9. The puncture needle gripping device according to claim 7, wherein a first rack is provided in the first substrate, a second rack that is slidable along the linear guide and is interlocked and connected to the first rack via a pinion is provided in a second substrate, and the second finger is mounted on the second substrate.

10. The puncture needle gripping device according to claim 8, wherein a first rack is provided in the first substrate, a second rack that is slidable along the linear guide and is interlocked and connected to the first rack via a pinion is provided in a second substrate, and the second finger is mounted on the second substrate.

11. A puncture robot comprising:
the puncture needle gripping device according to claim 1 provided at a distal end of a longitudinal arm,
wherein a target in a patient' body is punctured by the puncture needle by moving the longitudinal arm forward in an axial direction of the puncture needle gripped by the puncture needle gripping device.

12. The puncture robot according to claim 11, wherein the arm is provided with a spare switch for performing a remote control.

13. The puncture robot according to claim 11, wherein the remote control is an emergency stop operation.

14. The puncture robot according to claim 12, wherein the remote control is an emergency stop operation.
